(19) United States
(12) Patent Application Publication
(10) Pub. No.: US 2017/0273916 A1
(43) Pub. Date: Sep. 28, 2017

(54) METHODS FOR TREATING AND PREVENTING NEURODEGENERATIVE DISEASES

(71) Applicant: YIN YU KUO, Taipei City (TW)

(72) Inventors: MAO TIEN KUO, Taipei City (TW); HUI LING TSENG, Taipei City (TW); WAN PING TSENG, Taipei City (TW); TAI LIN TSENG, Taipei City (TW); YIN YU KUO, Taipei City (TW)

(21) Appl. No.: 15/465,098

(22) Filed: Mar. 21, 2017

(30) Foreign Application Priority Data
Mar. 25, 2016 (TW) 105109574

(51) Int. Cl. A61K 31/122; A61K 36/07

(52) U.S. Cl. CPC A61K 31/122; A61K 36/07

(57) ABSTRACT

The present invention provides a method for treating and preventing neurodegenerative diseases, comprising administering an effective amount of a compound isolated from Antrodia camphorate, represented by formula (I), to a subject in need thereof;

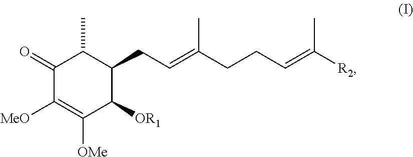

wherein R₁ is a hydrogen atom or an acetyl group, and R₂ is

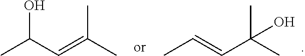

### CROSS-REFERENCE TO RELATED APPLICATION

This application claims the priority of Taiwanese patent application No. 105109574, filed on March 25, 2016, which is incorporated herewith by reference.

### BACKGROUND OF THE INVENTION

### 1. Field of the Invention

The present invention relates to a method for treating and preventing neurodegenerative diseases.

### 2. The Prior Arts

Antrodia camphorata is also called Chang-Zhi, Niu Chang-Zhi, red camphor mushroom and the like, which is a perennial mushroom belonging to the order Aphyllophorales, the family Polyporaceae. It is an endemic species in Taiwan growing on the inner rotten heart wood wall of Cinnamomum kanehirae Hay. Cinnamoum kanehirai Hay is rarely distributed and being overcut unlawfully, which makes Antrodia camphorata growing inside the tree in the wild became even rare. The price of Antrodia camphorata is very expensive due to the extremely slow growth rate of natural Antrodia camphorata that only grows between Junes to October.

The fruiting bodies of Antrodia camphorata are perennial, sessile, hard and woody, which exhales strong smell of sassafras (camphor aroma). The appearances are various with plate-like, bell-like, hoof-like, or tower-like shapes. They are reddish in color and flat when young, attached to the surface of wood. Then the brims of the front end become little curled tilted and extend to the surroundings. The color turns to be faded red-brown or cream yellow brown, with ostioles all over. This region is of very high medical value.

In traditional Taiwanese medicine, Antrodia camphorata is commonly used as an antidotal, liver protective, anti-cancer drug. Antrodia camphorata, like general edible and medicinal mushrooms, is rich in numerous nutrients including polysaccharides (such as β-glucosan), triterpenoids, superoxide dismutase (SOD), adenosine, proteins (immunoglobulins), vitamins (such as vitamin B, nicotinic acid), trace elements (such as calcium, phosphorus and germanium and so on), nucleic acid, agglutinin, amino acids, steroids, lignins and stabilizers for blood pressure (such as antodia acid) and the like. These physiologically active ingredients are believed to exhibit effects such as: anti-tumor activities, increasing immuno-modulating activities, anti-allergy, anti-bacteria, anti-high blood pressure, decreasing blood sugar, decreasing cholesterol and the like.

Triterpenoids are the most studied component among the numerous compositions of Antrodia camphorata. Triterpenoids are the summary terms for natural compounds, which contain 30 carbon atoms with the pent acyclic or hex acyclic structures. The bitter taste of Antrodia camphorata is from the component of triterpenoids. Three novel ergostane-type triterpenoids (antcin A, antcin B, antcin C) were isolated by Cherng et al. from the fruiting bodies of Antrodia camphorata (Cherng, I. H., and Chiang, H. C. 1995. Three new triterpenoids from Antrodia cinnamomea. J. Nat. Prod. 58:365-371). Three new compounds zhankuic acid A, zhankuic acid B and zhankuic acid were extracted from the fruiting bodies of Antrodia camphorata with ethanol by Chen et al. (Chen, C. H., and Yang, S. W. 1995. New steroid acids from Antrodia cinnamomea, -a fungus parasitic on Cinnamomum micranthum. J. Nat. Prod. 58:1655-1661). In addition, Cherng et al. also found three other new triterpenoids from the fruiting bodies of Antrodia camphorata, which are sesquiterpene lactone and 2 biphenyl derived compounds, 4,7-dimethoxy-5-methy-1,3-benzodioxole and 2,2′,5,5′-teramethoxy-3,4,3′,4′-bi-methylenedioxy-6,6′-dimethylbiphenyl (Chiang, H. C., Wu, D. P., Cherng, I. W., and Ueng, C. H. 1995. A sesquiterpene lactone, phenyl and biphenyl compounds from Antrodia cinnamomea. Phytochemistry. 39:613-616). In 1996, four novel ergostane-type triterpenoids (antcins E and F and methyl antcinates G and H) were isolated by Cherng et al. with the same analytic methods (Cherng, I. H., Wu, D. P., and Chiang, H. C. 1996. Triteroenoids from Antrodia cinnamomea. Phytochemistry. 41:263-267). And two ergostane related steroids, zhankuic acids D and E together with three lanosta related triterpenes, 15 alpha-acetyl-dehydrosulphurenic acid, dehydroeburicoic acid, dehydrosulphurenic acid were isolated by Yang et al. (Yang, S. W., Shen, Y. C., and Chen, C. H. 1996. Steroids and triterpenoids of Antrodia cinnamomea—a fungus parasitic on Cinnamomum micranthum. Phytochemistry. 41:1389-1392).

Alzheimer's disease (AD) is a chronic neurodegenerative disease that usually starts slowly and gets worse over time. It is the cause of 60% to 70% of cases of dementia. The most common early symptom is difficulty in remembering recent events (short-term memory loss). As the disease advances, symptoms can include problems with language, disorientation (including easily getting lost), mood swings, loss of motivation, not managing self-care, and behavioral issues. The cause of Alzheimer's disease is poorly understood. In 2015, there were approximately 48 million people worldwide with AD. It most often begins in people over 65 years of age, although 4% to 5% of cases are early-onset Alzheimer's which begin before this. It affects about 6% of people 65 years and older. In developed countries, AD is one of the most financially costly diseases. Alzheimer's has no current cure, but treatments for symptoms are available and research continues.

The exact causes of Alzheimer's disease are still unknown. Amyloid plaques, neurofibrillary tangles, and genetics are all thought to play a role in causing Alzheimer's disease. Amyloid plaques are dense, mostly insoluble clumps of protein fragments. The aggregation may cause oxidative stress and inflammation to damage the brain's nerve cells. Thus, anti-oxidation is highly related to prevention of AD Alzheimer's disease. PC12 cells have been widely used as a model for Alzheimer'sdisease differentiation.

### SUMMARY OF THE INVENTION

A primary objective of the present invention is to provide a method for treating and preventing neurodegenerative diseases.

In order to achieve the foregoing objective, the present invention provides a method for treating and preventing neurodegenerative diseases. The method comprises administering an effective amount of a compound isolated from Antrodia camphorate, represented by formula (I), to a subject in need thereof;

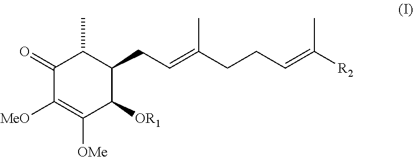

wherein R₁ is a hydrogen atom or an acetyl group, R₂ is

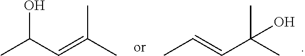

Preferably, wherein R₁ is a hydrogen atom, R₂ is

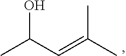

and the compound is represented by formula (II):

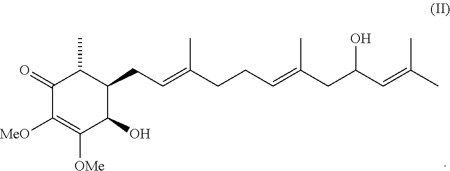 [SMILES: COC1=C(OC)[C@H](O)[C@H](C/C=C(\C)CC/C=C(\C)CC(O)C=C(C)C)[C@@H](C)C1=O]

Preferably, wherein R₁ an acetyl group, R₂ is

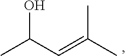

and the compound is represented by formula (III):

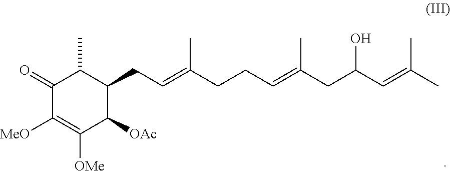 [SMILES: COC1=C(OC)[C@H](OC(C)=O)[C@H](C/C=C(\C)CC/C=C(\C)CC(O)C=C(C)C)[C@@H](C)C1=O]

Preferably, wherein R₁ is a hydrogen atom, R₂ is

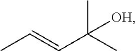

and the compound is represented by formula (IV):

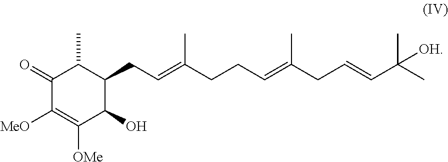 [SMILES: COC1=C(OC)[C@H](O)[C@H](C/C=C(\C)CC/C=C(\C)C/C=C/C(C)(C)O)[C@@H](C)C1=O]

### BRIEF DESCRIPTION OF THE DRAWINGS

FIGS. 1A-1E show viability of PC12 cells treated with extracts (ANCA and ANCA-D) and compounds (Antrocamol LT1, LT2, and LT3).

FIGS. 2A-2E show therapeutic effect of extracts (ANCA and ANCA-D) and compounds (Antrocamol LT1, LT2, and LT3) on AD cell model.

FIGS. 3A-3E show prophylactic effect of extracts (ANCA and ANCA-D) and compounds (Antrocamol LT1, LT2, and LT3) on AD cell model.

### DETAILED DESCRIPTION OF THE PREFERRED EMBODIMENT

The accompanying drawings are included to provide a further understanding of the invention, and are incorporated in and constitute a part of this specification.

### Methods and Materials

### 1.1 Experimental Material

Extracts (ANCA, ANCA-D) and compounds (Antrocamol LT1, LT2, LT3) are provided by Lantyng biotechnology corp. and stored at −20° C.

Antrodia camphorate Extracts

Antrodia camphorata fruiting bodies, mycelium or their mixture were provided (1.0 kg) and then extracted twice with an 10-fold ethanol solution to obtain two ethanol extracts. The ethanol extracts were concentrated to yield 230 g crude extract (LE-E). The crude extract was extracted three times with dichloromethane/water (1:1) to form a dichloromethane layer (LT-E-D, 102.6 g) and a water layer (LT-E-W, 127.4 g). Dichloromethane layer (6.0 g) was loaded to a layered silica gel column with hexane/dichloromethane (1:4), dichloromethane, and methanol/dichloromethane (5:95) to yield four layers, respectively ANCA-E-D-1, ANCA-E-D-2, ANCA-E-D-3, and ANCA-E-D-4 (Fitoterapia Volume 102, April 2015, Pages 115-119).

Antrocamol LT1, Antrocamol LT2, Antrocamol LT3 compounds

Antrocamol LT1, Antrocamol LT2, Antrocamol LT3 compounds were three compounds discovered by applicants, and purification process and of three compounds were disclosed in the previous application. These details are not described repeatedly. Their chemical formulas are disclosed as below:

### Antrocamol LT1

Antrocamol LT1 is represented by formula (II):

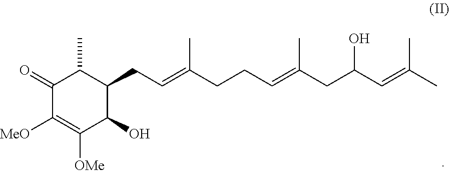 [SMILES: COC1=C(OC)[C@H](O)[C@H](C/C=C(\C)CC/C=C(\C)CC(O)C=C(C)C)[C@@H](C)C1=O]

Antrocamol LT1 was a transparent aqueous product, the molecular formula was determined as: C₂₄H₃₈O₅; 4-hydroxy-5-[9-hydroxy-3,7,11-trimethyldodeca-2,6,10-trienyl]-2,3-dimethoxy-6-methyl-cyclohex-2-enone; molecular weight: 406.

### Antrocamol LT2

Antrocamol LT2 is represented by formula (III):

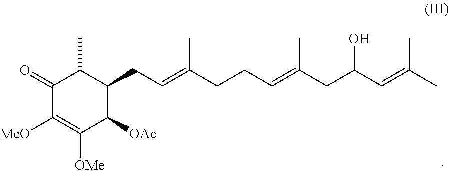 [SMILES: COC1=C(OC)[C@H](OC(C)=O)[C@H](C/C=C(\C)CC/C=C(\C)CC(O)C=C(C)C)[C@@H](C)C1=O]

Antrocamol LT2 was a transparent aqueous product, the molecular formula was determined as: C₂₆H₄₀O₆; 4-acetoxy-5-[9-hydroxy-3,7,11-trimethyldodeca-2,6,10-trienyl]-2,3-dimethoxy-6-methyl-cyclohex-2-enone; molecular weight: 448.

### Antrocamol LT3

Antrocamol LT3 was a colorless liquid product, and it was analyzed and found that its molecular formula was C₂₄H₃₈O₅ with a molecular weight of 448. The complete name for this compound was called(4R,5R,6R)-4-hydroxy-5-[(2E,6E,9E)-11-hydroxy-3,7,11-trimethyldodeca-2,6,9-trienyl]-2,3-dimethoxy-6-methylcyclohex-2-enone.

Antrocamol LT3 is represented by formula (IV):

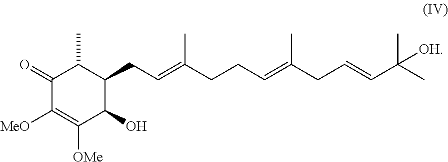 [SMILES: COC1=C(OC)[C@H](O)[C@H](C/C=C(\C)CC/C=C(\C)C/C=C/C(C)(C)O)[C@@H](C)C1=O]

Antrocamol LT1, Antrocamol LT2 and Antrocamol LT3 has a similar main structure, the general formula can be represented by formula (I):

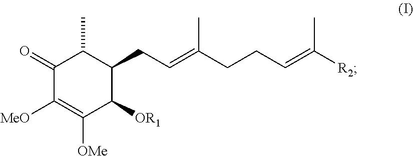

wherein R₁ is a hydrogen atom or an acetyl group, R₂ is

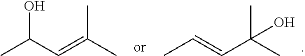

For detailed information about extraction and purification of three aforementioned compounds from Antrodia camphorata, they can be referred to the inventor's previous relative applications (U.S. Ser. No. 14/880,695, U.S. Ser. No. 13/960,764 and U.S. Ser. No. 14/624,015). In the present invention, the experiments with three compounds are conducted in order to determine their therapeutic and prophylactic influences for neurodegenerative neurodegenerative diseases, and to study that whether those compounds have medical potentials for treating and preventing Alzheimer's disease.

### Cell Culture

PC12 cell line is derived from a pheochromocytoma of the rat adrenal medulla. PC12 cell line used herein is purchased from Shanghai institutes for biological sciences (200031, Shanghai, China).

### Experimental Agents

RPMI 1640 medium, WISENT; Fetal bovine serum (FBS), WISENT; Heat-inactivated Horse serum, Gibco; Penicillin-Streptomycin (100X), Beyotime; Dimethyl Sulfoxide (DMSO), Sunshine Bio; Trypsin 1:250, Sunshine Bio; EDTA 2Na, Sunshine Bio; Phosphate buffered saline (PBS), Beyotime; NGF 2.5S Native Mouse Protein, Invitrogen; Amyloid β Protein Fragment 1-40, Sigma-Aldrich; Trifluoroacetic acid 99%, TCI; CCK-8 cell viability assay kit, Beyotime.

### Experimental Instruments

Forma™ Series 3 Water Jacketed CO₂ Incubator, Thermo; MSC-Advantage™ Class II Biological Safety Cabinets, Thermo; ENTRIS64-1S Analytical Balance, Sartorius; HH-4 Digital Water bath; SB25-12DT Ultrasonic cleaning; MLS-3780 Autoclave, SANYO; DHG-9423A Electric oven thermostat blast; Bio-MEDICAL HYCD-282 Ultra-Low Temperature Freezers, Haler; Allegra X-15R Benchtop Centrifuge, Beckman; Eclipse TS100 Inverted Routine Microscope, Nikon; Infinite M1000 PRO Microplate Readers, TECAN. Although the present invention has been described with reference to the preferred embodiments thereof, it is apparent to those skilled in the art that a variety of modifications and changes may be made without departing from the scope of the present invention which is intended to be defined by the appended claims.

### Cell Culture for PC12 Cells

PC12 cells were cultured in RPMI 1640 medium (5% fetal bovine serum, 10% heat-inactivated horse serum, 2 mM glutamine, 50 U/mL penicillin, 50 mg/mL streptomycin), at 37° C., 5% CO₂, subculture twice a week.

PC12 cells need to be cultured in medium containing Nerve growth factor (NGF) 2.5S, (100 ng/mL) in advance for NGF-induced neuronal differentiation.

### Treatment of Extracts and Compounds on PC12 Cultures

Cell Counting Kit-8(CCK-8) allows sensitive colorimetric assays for the determination of cell viability in cell proliferation and cytotoxicity assays. Dojindo's highly water-soluble tetrazolium salt, WST-8, is reduced by dehydrogenase activities in cells to give a yellow-color formazan dye, which is soluble in the tissue culture media. The amount of the formazan dye, generated by the activities of dehydrogenases in cells, is directly proportional to the number of living cells. The detection sensitivity of CCK-8 is higher than the other tetrazolium salts such as MTT, XTT, MTS or WST-1.

To optimize the exemplary extracts (ANCA, ANCA-D) and compounds (Antrocamol LT1, LT2, LT3) concentration for NGF-induced PC12 neural model, titration experiments were performed to determine the IC₅₀ values of extracts and compounds. 5 x 10⁴ NGF-induced PC12 cells were cultured in 96-well dish for 24 hr. Then the cells were cultured in medium with 1% fetal bovine serum, 2% heat-inactivated horse serum, 2 mM glutamine, 50 U/mL penicillin, 50 mg/mL streptomycin RPMI 1640 medium, then various concentrations of extracts [ANCA, ANCA-D (0.064, 0.32, 1.6, 8, 40, 200 μg/mL)] and compounds [Antrocamol LT1, LT2, LT3 (0.032, 0.16, 0.8, 4, 20, 100 μg/mL) were added individually and incubated for 24 hr. After treatment, the CCK-8 solution was added to each well of plates and incubates the plates for 4 hr in the incubator. The concentration of the formazan product was determined spectrophotometrically at an absorbance wavelength 450 nm and cell viability was expressed as a percentage of the corresponding control.

### Treatment of Extracts and Compounds on Aβ-Induced PC12 Cell Model

β-Amyloid 1-40 (Aβ) was dissolved in 0.1% (v/v) trifluoroacetic acid (TFA) in water at 10 mg/ml and stored at −20° C. as a stock solution. Af3 was diluted to 0.5 mg/ml with phosphate-buffered saline solution (PBS, without Ca2+) and aggregate at 25° C. for 48 hr before use. To investigate the therapeutic effect of extracts (ANCA, ANCA-D) and compounds (Antrocamol LT1, LT2, LT3) on Alzheimer disease (AD) cell model in vitro, cultures were pretreated with Aβ for 24 h and then exposed to freshly prepared extracts (ANCA, ANCA-D) and compounds (Antrocamol LT1, LT2, LT3) for 24 hr before cell viability measurements. In contrast, to investigate the prophylactic effect of extracts (ANCA, ANCA-D) and compounds (Antrocamol LT1, LT2, LT3) on AD cell model, cultures were pretreated with extracts (ANCA, ANCA-D) and compounds (Antrocamol LT1, LT2, LT3) for 24 h and then exposure to aggregated Aβ for 24 h before cell viability measurements. The cell viability was estimated by below formulation:

Cell viability (%)=(Sample-Blank)/(Control-Blank)×100%

### Experiment Results

### Treatment of Extracts and Compounds on PC12 Cultures

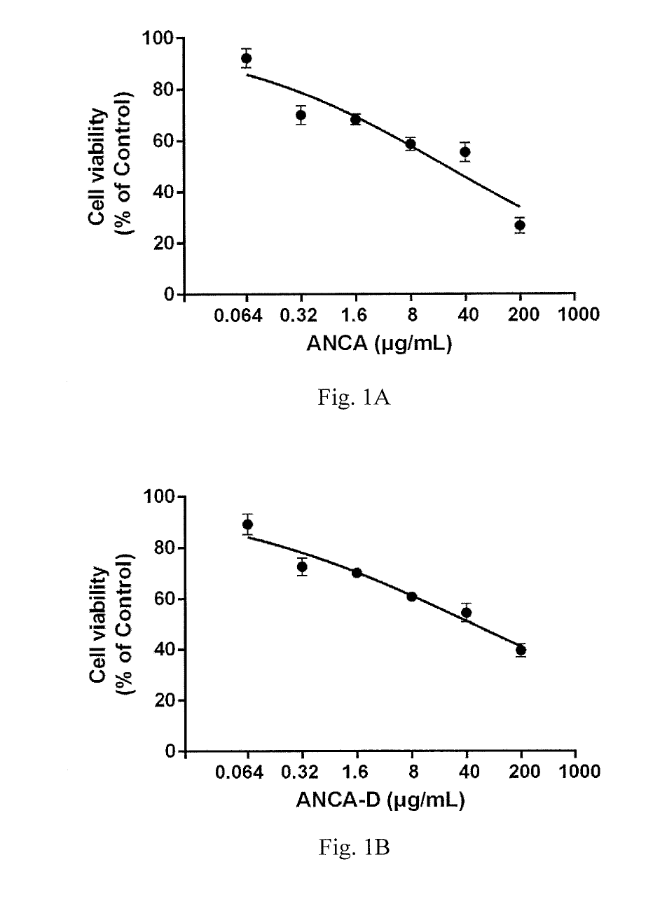

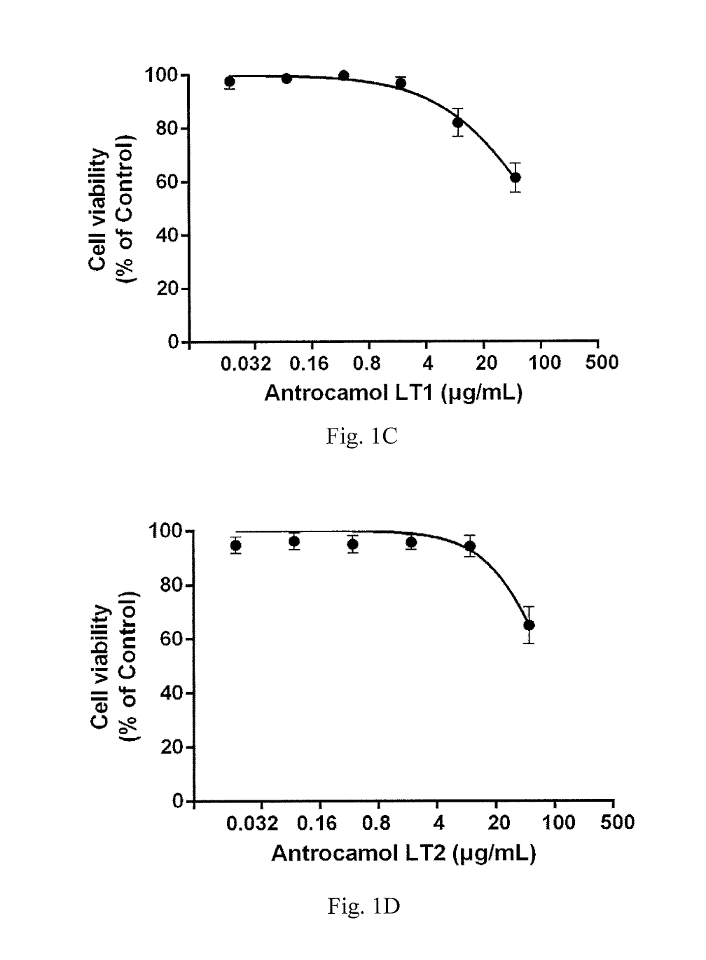

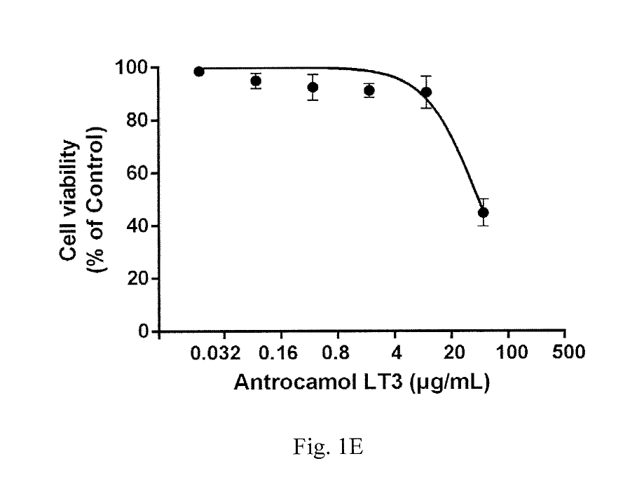

IC₅₀ values of extracts ANCA and ANCA-D are determined to be 22.91, 49 μg/mL (FIGS. 1A-1B). IC₅₀ values of the compounds (LT1, LT2, LT3) are determined to be 178.5, 168.5, 105.5 μg/mL (FIGS. 1C-1E).

### Treatment of Extracts and Compounds on Aβ-Induced PC12 Cell Model

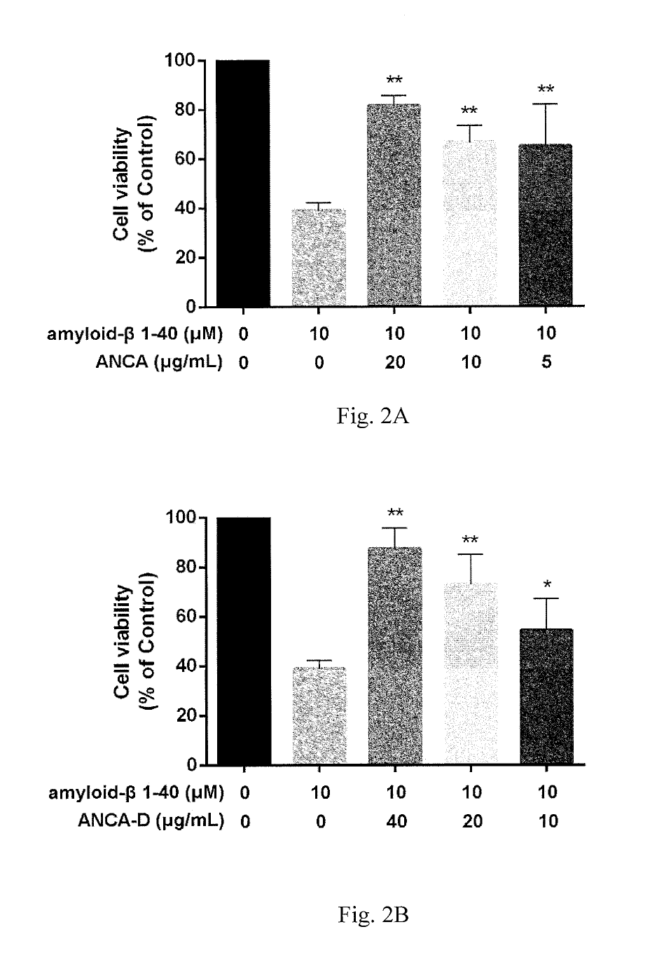

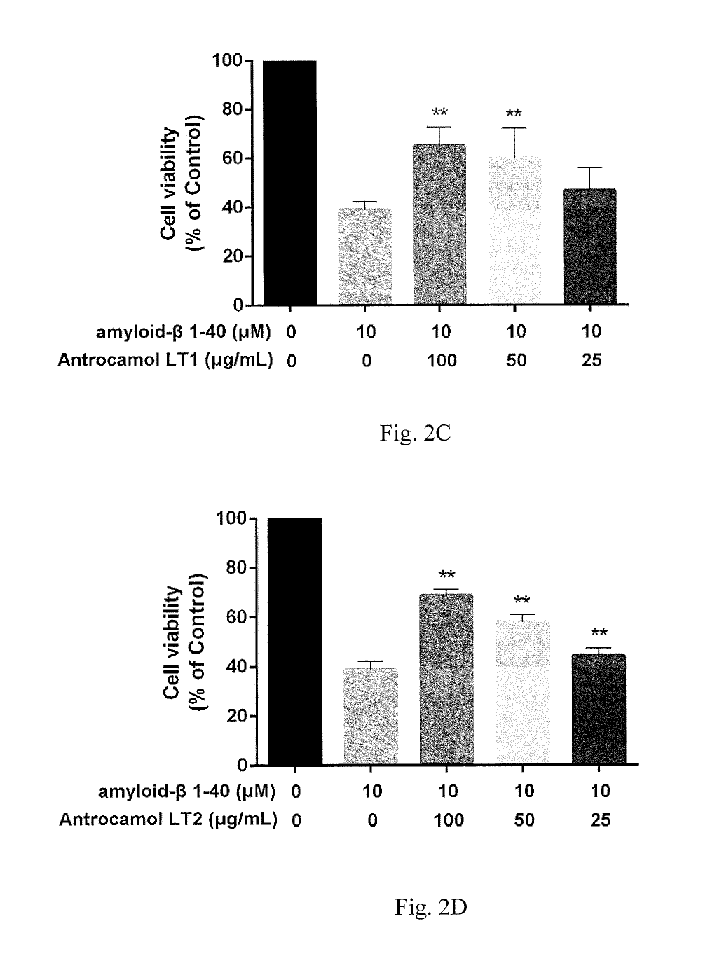

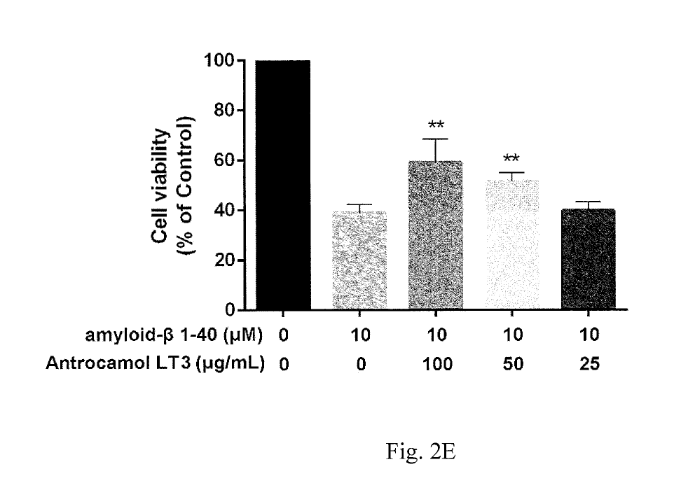

Regarding therapeutic effect of extracts (ANCA, ANCA-D) and compounds (Antrocamol LT1, LT2, LT3) on AD cell model, the cell viability results indicates that all extracts and compounds can significantly inhibited Aβ-induced damage and improved the cell viability (FIGS. 2A-2E). Particularly, treating effect of extracts ANCA and ANCA were the best. Compounds LT1 and LT2 also had a significant inhibitory effect at 100 μg/mL and 50 μg/mL), however the inhibitory effect were weak at 25 μg/mL. Similarly, compound LT3 had a significant inhibitory effect at 100 μg/mL and 50 μg/mL), and the inhibitory effect was weak at 25 μg/mL.

Regarding therapeutic effect of extracts (ANCA, ANCA-D) and compounds (Antrocamol LT1, LT2, LT3) on AD cell model, the cell viability results indicates that all extracts and compounds can significantly inhibited Aβ-induced damage and improved the cell viability (FIGS. 2A-2E). Particularly, treating effect of extracts ANCA and ANCA were the best. Compounds LT1 and LT2 also had a significant inhibitory effect at 100 μg/mL and 50 μg/mL), however the inhibitory effect were weak at 25 μg/mL. Similarly, compound LT3 had a significant inhibitory effect at 100 μg/mL and 50 μg/mL), and the inhibitory effect was weak at 25 μg/mL.

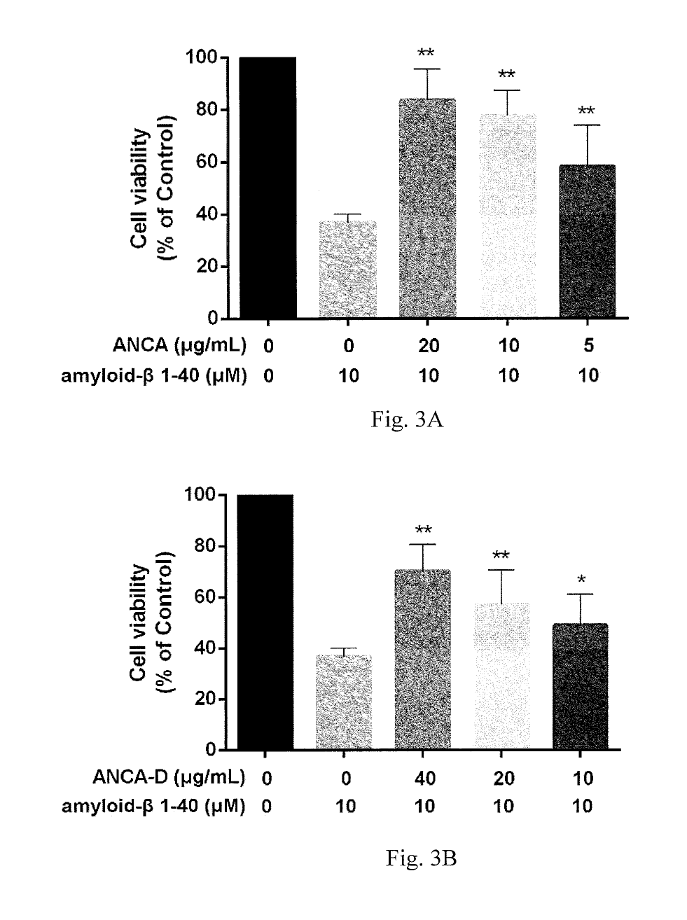

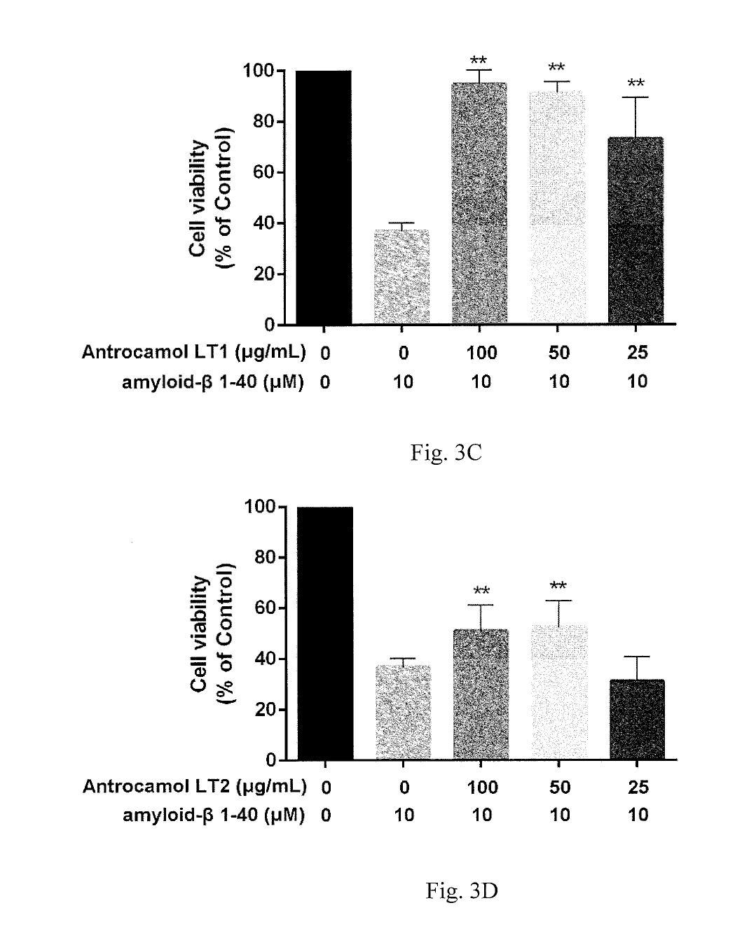

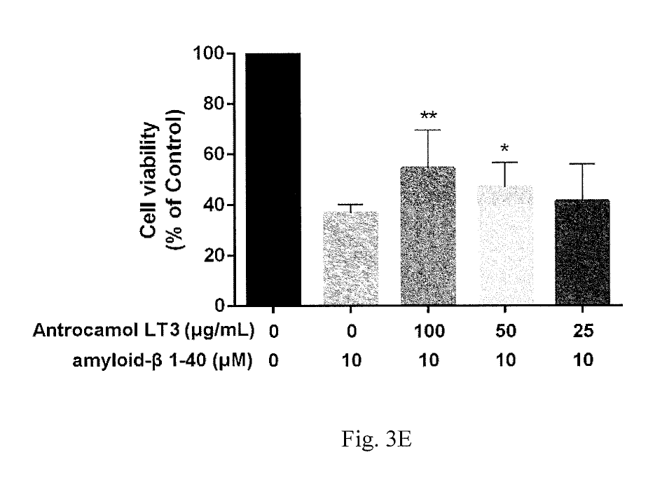

Further, regarding prophylactic effect of extracts (ANCA, ANCA-D) and compounds (Antrocamol LT1, LT2, LT3) on AD cell model, the cell viability results indicates that all extracts and compounds exhibits a significant impact in preventing (reducing the risk) cells from Aβ-induced neuronal damage (FIGS. 3A-3E). Particularly, prophylactic effect of extracts ANCA and ANCA were significant. Compounds LT1 (100 μg/mL, 50 μg/mL, 25 μg/mL) has a better prophylactic effect than extracts. However, compounds LT2 and LT3 exhibit a weak preventing effect.

According to these results, extracts (ANCA, ANCA-D) and compounds (Antrocamol LT1, LT2, LT3) indeed exhibit the potential to treat and prevent neurodegenerative diseases, despite their mechanism still unclear and need further research.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for treating and preventing neurodegenerative diseases, comprising administering an effective amount of a compound isolated from Antrodia camphorate, represented by formula (I), to a subject in need thereof; 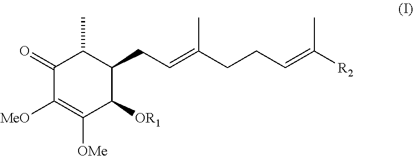
wherein R₁ is a hydrogen atom or an acetyl group, and R₂ is

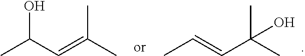

2. The method as claimed in claim 1, wherein R₁ is a hydrogen atom, R₂ is 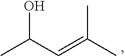
and the compound is represented by formula (II):

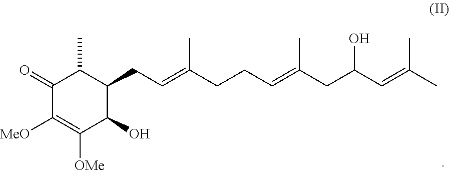 [SMILES: COC1=C(OC)[C@H](O)[C@H](C/C=C(\C)CC/C=C(\C)CC(O)C=C(C)C)[C@@H](C)C1=O]

3. The method as claimed in claim 1, wherein R₁ an acetyl group, R₂ is 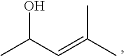
and the compound is represented by formula (III):

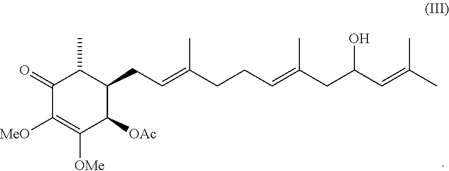 [SMILES: COC1=C(OC)[C@H](OC(C)=O)[C@H](C/C=C(\C)CC/C=C(\C)CC(O)C=C(C)C)[C@@H](C)C1=O]

4. The method as claimed in claim 1, wherein R₁ is a hydrogen atom, R₂ is 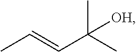
and the compound is represented by formula (IV): 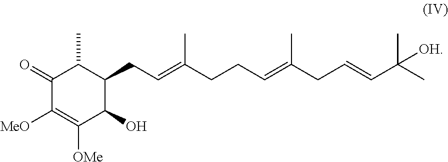 [SMILES: COC1=C(OC)[C@H](O)[C@H](C/C=C(\C)CC/C=C(\C)C/C=C/C(C)(C)O)[C@@H](C)C1=O]
